(19) United States
(12) Patent Application Publication
(10) Pub. No.: US 2019/0069932 A1
(43) Pub. Date: Mar. 7, 2019

(54) SPINAL MULTI-LEVEL INTERSEGMENTAL STABILIZATION SYSTEM AND METHOD FOR IMPLANTING

(71) Applicant: LinkSPINE, Inc., Columbia, MD (US)

(72) Inventor: Christopher D. CHAPUT, Temple, TX (US)

(73) Assignee: LinkSPINE, Inc., Columbia, MD (US)

(21) Appl. No.: 15/766,553

(22) PCT Filed: Oct. 10, 2016
(86) PCT No.: PCT/US2016/056290

§ 371 Date: Apr. 6, 2018

Related U.S. Application Data
(60) Provisional application No. 62/239,766, filed on Oct. 9, 2015.

(51) Int. Cl. A61B 17/70

(52) U.S. Cl. CPC A61B 17/7064; A61B 17/7044; A61F 2/4405; A61B 17/7037; A61B 17/701

(57) ABSTRACT

A multi-level intersegmental stabilization implant for a facet joint of a first vertebra includes a lower retainer module having a transfacetal fastener and an upper retainer module having a second fastener configured for fastening through the pedicle of an upper vertebra. The lower and upper retainer modules are connected by an elongated carrier element dimensioned such as to span across at least two vertebrae. The transfacetal fastener is configured as a screw having a head and a shaft ending with a tip, the shaft being provided with a thread at least in a region near its tip such that it engages exclusively the lower section of the facet joint, wherein a washer is provided co-operatively connected with the transfacetal fastener, the washer being configured for bearing on an outer surface of the upper section of the facet joint.

### CROSS-REFERENCE TO RELATED APPLICATIONS

This application is a national stage application under 35 USC 371 of International Application no. PCT/US2016/056290 filed Oct. 10, 2016, which claims priority to U.S. Provisional Patent Application No. 62/239,766, filed Oct. 9, 2015, the entire contents of each of which are incorporated herein by reference in its entirety.

### FIELD OF INVENTION

The invention relates to a spinal stabilization system and implanting method for multilevel intersegmental application.

### BACKGROUND OF THE INVENTION

In the human body, the spinal column is a structural element of the human skeleton. It is comprised of a plurality of articulating vertebrae, which are arranged one above another divided by intervertebral discs. The discs allow for transfer of load between the vertebrae while the facet joints are the structure that connects the vertebrae one to another. Each vertebra has two sets of facet joints. One pair faces upward (superior articular facet) and one downward (inferior articular facet) with one joint on each side. The wear of the articulated connection (facet joints) of two neighbouring vertebrae may lead to a restricted movement, pain or even loss of mobility. This is one of various sources of pain or mobility issues that can be treated with this method. Various approaches have become known for treatment. In particular, a definite improvement can be achieved by stabilizing the facet joint (facet joint fusion) or stabilizing the spine connecting the pedicles of at minimum two adjacent vertebral bodies.

For the facet joint fusion a fusion implant is known which comprises two long bone screws each of which is screwed through both of the facets forming a facet joint (WO 2012/072733 A1). This known fusion implant offers the advantage of relatively simple implantability because it only has small dimensions and therefore can be implanted even in minimally invasive surgery. However, this known fusion implant requires a relatively strong and intact bone structure of the vertebral body.

Quite often the adjacent structure is not strong enough. In such a case further stabilization is required, necessitating extensive stabilization systems. Their implantation may require massively invasive surgery. This is stressful for the patient. Further, quite often it cannot be determined in advance whether such stabilization would be indeed required. Intra-operative switch-over to such a system adds to complexity and therefore risk of the whole surgery process.

### SUMMARY OF THE INVENTION

An objective according to aspects of the invention is to provide an improved implant which is more flexible in usage. Given a minimum of three adjacent vertebral bodies, a method according to some embodiments allows to stabilize the segments using the superior facet joint of the lowest vertebra, the inferior facet joint of the middle vertebra together with the pedicle of the superior level. It may comprise a retainer module and a transfacetal fastening means co-operatively engaged to a washer having a polyaxial seat.

According to aspects of the invention, a simplified multi-level-intersegmental stabilization system is provided. Given a minimum of three vertebrae in a target area, a multi-level intersegmental fixation is achieved using a lower retainer module having a transfacetal fastening device to lock the facet joint comprised of the superior facet of an inferior vertebra and the inferior facet of a middle vertebra and an upper retainer module comprising of a second fastening means configured for a fastening to the pedicles of a superior vertebra. The transfacetal fastening means has a head and a shaft traversing the inferior facet of the middle vertebra and upper facet of the inferior vertebra. The lower and upper retainer modules are connected by an elongated carrier element (rod) that is dimensioned such as to span across at least the middle and superior vertebrae of the target area comprising said at least three adjacent vertebrae. According to the invention, the transfacetal fastening means is configured as a screw having a head and a shaft ending with a tip, the shaft being provided with a thread at least in a region near its tip such that it engages exclusively the lower section of the facet joint, and it co-operates with a washer having a hole, a pressure side and an opposing seating side, the hole being configured for receiving the shaft of the screw, the pressure side being configured for bearing on an outer surface of the upper section of the facet joint and the seating side being configured to being abutted by the screw such that a compression force exerted by the screw is transferred through the washer onto the facet joint, wherein the washer provides a polyaxial seat for the screw.

The term “elongated carrier element” relates to an extension element that is reaching predominantly in an upward direction, but is also usable in horizontal direction. The direction indications refer to an implanted state of the implant.

The term “span” means to reach that far over a vertebra so that the second fastening means can engage an upper neighbouring vertebra (“cantilevered”), without being fixated to a middle vertebra (if any).

Given at least three vertebral bodies, the invention according to some embodiments is based on the idea to provide a combination of a facet joint immobilization implant which connects the lower and middle vertebra together with a stabilizing implant which is anchored to the superior vertebra. By a co-operation of the transfacetal screw and the washer with its polyaxial seating for the screw, a double effect is achieved, namely a fusion of the facetal joint and creation of a stable anchoring for the implant in a multi-level configuration. The lower (far) portion of the facet joint is pulled towards the head of the screw by the force exerted by the thread when threaded in, and simultaneously the head of the screw being seated on the polyaxial seat of the washer presses the washer and the upper (near) portion of the facet joint downwards toward the tip of the screw, thereby exerting a strong compression force on the facet joint. This provides for a strong fixation and, as a result, immobilization of the facet joint.

Moreover, the stabilization system with the addition of a ball and a retainer module having a locking polyaxial joint (tulip) on the top the transfacetal fastening element makes double use of this element, thereby reducing the total number of fastening element required. Conventionally, given three vertebral bodies, six bone screws were required for providing a multi-level stabilization, and the invention according to some embodiments just needs four.

In addition, the polyaxial seating of the screw on the washer allows for a compact configuration of the implant. It allows for a highly oblique angle of the transfacetal screw in respect to the elongated carrier element. By virtue of this, an instrument used to mount and tighten the transfacetal fastening means is attached near to the center of the multi-fusion implant. Thereby, a rather small access hole will be sufficient for implantation, as opposed to conventional multi-level implants which lack such an oblique configuration of the screw and consequently require more space for mounting, i.e. a much bigger access hole and consequently wound to the patient. This is a considerable advantage in terms of surgery success.

This advantage is further increased by an angled configuration of the fastening means, those of the lower and upper support, such that their trajectories are converging in a direction away from the vertebrae. Thereby, the same small access hole will be sufficient for mounting and tightening of the fastening screws to be used in the upper support. By virtue of such a converging arrangement of the fastening means, space requirements for the implantation of the multi-level implant according to the invention are minimized. It was found that space savings and robust anchoring is best balanced for a converging angle of preferably at least 60°.

Preferably, the elongated carrier element is shorter than a distance between the first and second vertebrae, preferably by at least half of a height of a vertebra.

Preferably, the elongated carrier element features a noncircular cross-section. Thereby, a preset angular orientation of the retainer module with respect to an axis of the elongated carrier element could be achieved. Thereby, any unwarranted rotating of the retaining module on the elongated carrier element is prevented. This is particularly useful if the elongated carrier element is shaped rodlike.

It is to be noted that another such washer may be provided for the upper retainer module. Providing of such a washer has the advantage of identical washer configuration in respect to the lower and upper retainer module. However, providing the additional washer for the upper retainer module is purely optional for the invention. Yet, for the sake of easier mounting and avoiding any risk of mounting the multi-level in an upside-down configuration, the upper and the lower retainer modules are configured identical. Thereby, the upper and lower end are interchangeable allowing a mounting of the implant even in an inverted (upside down) configuration. As a result, any negative impact which may otherwise result from a wrong orientation is avoided, in the interest of patient safety.

Preferably, the retainer module comprises a lockable poly-axial joint. Thereby, the orientation of the (transfacetal or secondary) fastening means could be adjusted in a wide range in order to effect fixation at the upper vertebra.

The lockable polyaxial joint preferably comprises a sleeve having a reduced width at its front, forming a polyaxial seat for the upward extension element. The range for said polyaxiality could be +50 degrees or more. Further, it is preferred that the sleeve comprises a tension cage in its interior and a pressing element, wherein the tension cage is configured to tiltably engage the head and the pressing element is configured to squeeze the tension cage for arresting of the head. Under a pressing force exerted by the pressing element, the tension cage which encloses the head is pressed against said head, thereby effecting a press-fit I which ensures a stable angle fixation.

The range of motion for polyaxial movement of the fastening means is preferably limited by a skirt surrounding the head portion. However, in order to provide a sufficient range of angular motion to the upward direction, the rim is preferably slanted in respect to a center axis of the sleeve.

Thereby, the range of angular motion is biggest in a direction facing away from the elongated carrier element, at the expense of the range of motion in the opposite direction) which is of no interest in such a configuration. As a result, the slanted orientation of the rim gives a favourable bias for the angular range of motion of said polyaxial attachment.

In a preferred embodiment, the shaft of the transfacetal screw is threaded in its tipward portion only. The tipward portion is that section of the shaft which is designed to engage the lower (far) section of the facet joint. As a consequence, by tightening of the transfacetal screw the lower section only (and not the upper section) is pulled upward. Alternatively, the shaft could be threaded along both, its tipward and its headward portion provided that its headward portion features a diameter which is smaller than a width of the bore through the upper section of the facet joint receiving the shaft. Thereby, the same effect is achieved, namely that the thread engages the lower section only.

In a particularly preferred embodiment, the implant is provided as a pair wherein the other implant is preferably a mirror image. Such a pair is particularly useful for implantation on either side of the spine, thereby providing a bilateral stabilization. The mirror image configuration ensures an optimum fit on either side. However, the mirror image configuration is not a must. The pair may comprise identical implants, too. Preferably, the other implant is arranged in a slanted position in its implanted state such that its fastening means are converging with respect to the fastening means of the implant. By virtue of the converging angle, a better stabilization against lateral forces can be achieved, thereby improving stabilization of the affected vertebrae in a lateral direction, while maintaining simplicity of the design and ease of implantation.

In a preferred embodiment, the transfacetal fastening means further comprises a super-head to form a stacked double head arrangement. The super-head is ball shaped and the head is preferably cylindrically or conically shaped. The ball shaping of the super-head allows a rather high degree of angular motion. Further, the ball shape is convenient for use and allows attachment of the top carrier in any position. Further, the ball shape is low in terms of danger of irritating surrounding tissue.

On another token, the super-head is polyaxially held in a second sleeve having a reduced width at its front. The second sleeve operates in a manner similar to the sleeve of the upward extension element, i. e. combining wide angular adjustability with strong fixation by a press fit. To this end, the second sleeve comprises a second tension cage in its interior and a second pressing element, wherein the second tension cage is configured to tiltably engage the super-head and the second pressing element is configured to squeeze the second tension cage to the super-head.

The sleeve preferably comprises at least one slot in its rear portion, the slot being configured for reception of the elongated carrier element, the elongated carrier element being preferably arrested by the second pressing element. By fixating of the elongated carrier element, a creation of a multi-level stabilization which jumps an intermediate vertebra and leaves it untouched is feasible.

The invention further relates to an arrangement of a sleeve and fixation element, like a screw. For further details, reference to the foregoing explanation is made.

The invention further relates to corresponding methods for implanting. Accordingly, a method for stabilizing a facet joint comprises the steps of opening an access hole providing access to a vertebra having a facet joint to be stabilized, forming a hole through the facet joint, the hole crossing an upper section of the facet joint and reaching into a lower section of the facet joint, positioning a washer on top of the hole, inserting a transfacetal fastening means through the washer into the hole, tightening the transfacetal fastening means such that the facet joint is immobilized, whereby a shaft of the transfacetal fastening means is polyaxially orientable in respect to the washer, forming a second hole in a pars portion of a superior vertebra, inserting a second fastening means, and closing the access hole, wherein the transfacetal fastening means and the second fastening means are each held in retainer module which are connected by a rigid, axially adjustable connection member, the transfacetal fastening means being polyaxially adjustable in its retainer module.

By this method, the implant as described above can be implanted. Additionally, this method requires a minimum size for the access hole only, thereby keeping the surgery wound small and aiding in fast recovery of the patient. The minimum size access hole is a particular advantage if the connecting member is configured such as to span over at least one intermediate vertebra.

Preferably, the transfacetal fastening means and the second fastening means are angled such that their tips diverge by an angle of at least 30°. Such a rather high diversion angle ensures that the opposite ends will be positioned in close proximity to each other. This is important since any tools for engaging the fastening means will be applied there. Hence, the tool access area is rather confined. Conventionally, in such a scenario a rather large access hole would be required, its size mirroring the combined height of the vertebrae concerned. According to the inventive method, this size can be reduced considerably about the height of one or two vertebra. As a result, an access being about the height of one vertebra would be sufficient to implant a multi-stage implant that engages three vertebrae. This is a huge advantage for the patient and his recovery.

For effecting an improved fusion of the facet joint, the upper and lower sections of the facet joint shall be compressed. To this end, the transfacetal fastening means are configured such that their thread interacts with the lower section only. This could be achieved in two ways. A first way is to employ a lag thread for the transfacetal fastening means which is configured such as to have threads in the portion interacting with the lower section only. As a result, no direct interaction of the thread w the upper section will occur, thereby increasing compressing efficiency. A second way is to have a full thread, but eliminating interaction with the upper section by giving it a larger bore. Thereby, the threads which may be a full thread will be engaged only with the (smaller) bore in the lower section, but not in the (too wide) bore in the upper section.

### BRIEF DESCRIPTION OF THE DRAWINGS

The invention will now be explained in reference to the enclosed drawing, which shows an advantageous sample embodiment. There are shown:

FIG. 1 is a side view of the implant position at the vertebrae of the spine;

FIG. 2a-c are lateral, rear and bottom view of the implant of FIG. 1;

FIG. 3a-b are embodiments of a washer;

FIG. 4a-b are side view and cross-section of the washer of FIG. 3b;

FIG. 5 is an exploded view of a retainer with a transfacetal screw;

FIG. 6 is a detail view of a slanted rim on the sleeve;

FIG. 7a-c are detail views of retainers with different screws;

FIG. 8a-e are views showing assembly of retainer and transfacetal screw, and

FIG. 9a-e are some steps showing implantation.

### DETAILED DESCRIPTION OF THE INVENTION

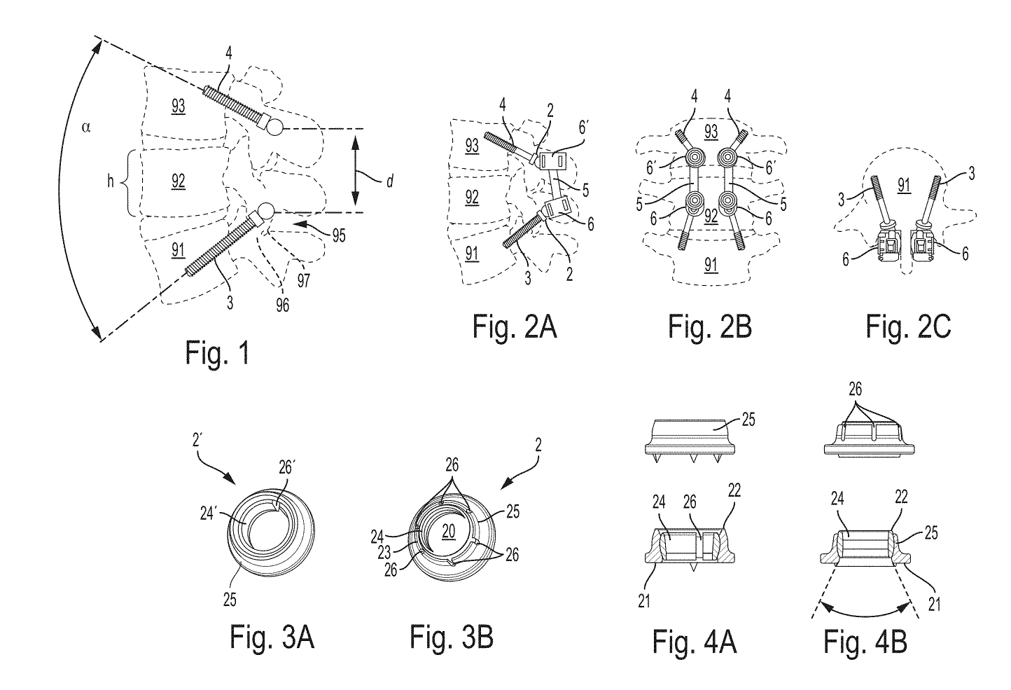

An embodiment of a fusion implant 1 comprises a lower and upper retainer module 6, 6′ and an elongated carrier element 5. In the lower retainer module 6, a transfacetal fastening means 3 is held. In the upper retainer module 6′, a second fastening means 4 is held. Further, a washer 2 is provided through which the screw 30 is routed. As it can be readily appreciated in the drawing of FIG. 1, the transfacetal fastening means 3 and second fastening means 4 are diverging at an angle α (the retainer modules 6, 6′ and the elongated carrier element 5 are omitted in FIG. 1 for clarity). Thereby, the heads of the transfacetal fastening means 3 and second fastening means 4 are located in close proximity (much closer than the positioning of the transfacetal fastening means 3 and second fastening means 4 at the vertebrae themselves), so that a rather small access hole will suffice. A small access hole means a small surgery wound and therefore lesser stress for the patient.

The transfacetal fastening means 3 comprise a screw having a shaft 30 with a thread 34, a head 31, and a tip 35. The shaft 30 is of sufficient length to traverse both, a lower and an upper section 96, 97 of a facet joint 95 of the two adjacent vertebrae 90 and 91. It comprises a thread 34 which may be cortical or cancellous thread, i.e. optimized for engaging cortical or cancellous bone structure, respectively. The thread may be configured as a full thread 34 as shown in FIG. 6 or a lag thread 34′ as shown in FIG. 7a), b). In the latter case, the thread 34′ is present in a tip-ward portion of the shaft 30 only, whereas the headward portion of the shaft 30 is threadless.

The washer 2 features a center bore 20 which receives the shaft 30 of the transfacetal fastening means 3. The washer 2 has a lower side acting as a pressure face 21 configured to bear on an outer surface of the vertebra 91, and an upper side acting as a seating 22 for the transfacetal fastening means 3. The seating is configured as a polyaxial seat 23 for the screw of the transfacetal fastening means 3. The length of the lag thread 34′ is dimensioned such as it will be placed completely in the lower section 96 of the facet joint 95.

For an improved transmission of force via the washer 2 to the facet joint 95, the seating is configured as a polyaxial seat 23. It comprises a spheroid ring 24 which is tiltably mounted in a complementary shaped receptable formed by a collar 25. The spheroid ring 24 and/or the collar 25 may be slotted. In an embodiment of the washer 2′ as shown in FIG. 3a the spheroid ring 24′ features a single slot 26′, whereas in the embodiment of the washer 2 as shown in FIG. 3b the spheroid ring 24 is unslotted and the collar 25 features six slots 26 around its perimeter. The spheroid ring 24 is placed such in the collar 25 that it is recessed referenced to the pressure face 21, thereby forming a free space 26 underneath of the spheroid ring 24. The free space 27 is dimensioned such as to allow a range of movement of at least 45° tilting angle for the spheroid ring 24. In a mounted state, the transfacetal fastening means 3 abuts with its head 31 on the spheroid ring 24 which will be tilted under the tensioning force of the transfacetal fastening means 3 such as to provide circumferential support for the head 31. By further tightening of the transfacetal fastening means 3, the head 31 will assisted by an optional slightly conical shape—arrest the angular position of the spheroid ring 24, thereby fixating the tilt angle. As a result, the facet joint is immobilized and secured.

By tightening of the transfacetal fastening means 3, the i lower section 96 of the facet joint 95 is pulled upward by the lag thread 34′ of the transfacetal fastening means 3, against the upper section 97 of the facet joint which is forced downwards under the pressure exerted by the washer 2 which acts as a counter support for the transfacetal fastening means 3 held in the retainer module 6. Thereby, both sections 96, 97 of the facet joint 95 are tightened against each other, thereby reliably immobilizing the facet joint 95. Alternatively, in the case of a full thread 34 a bore for in the upper section 97 of the facet joint 95 is made wider than an outer diameter of the thread 34, thereby it is also ensured that the full thread 34 will engage the lower section 96 of the facet joint 95 only.

The head 31 may have an additional thread 36 on its outer circumference which is configured to wedge against the spheroid ring 24 in its collar 25 in order to lock a desired angular position. Thereby, the tilt angle for the transfacetal fastening means 3 could be adjusted and securely fixed for a secure immobilization of the facet joint between the vertebrae 90 and 91.

The head 31 of the transfacetal fastening means 3 is part of a stacked double head arrangement which features at its end an additional head superimposed on the head 31, this additional head being termed a ball-shaped super-head 32.

On the super head 32, an elongated carrier element 5 is affixed. Said elongated carrier element 5 is linked to the transfacetal fastening means 3 by means of the retainer module 6 having a lockable polyaxial joint. At a far end of the elongated carrier element 5, a cortical bone screw acting as a second fastening means 4 is located which is configured for an oblique fastening to a cortical structure of an upper vertebra 93. It features a pointed tip configured for breaking the surface of upper vertebra 93 in order to gain access for better stabilization. At the other end opposite to the tip, a ball shaped head similar to the super-head 32 is provided. It is to be retained by a retainer module 6′ configured similar to the retainer module 6 which will be described below.

The lockable polyaxial joint in the retainer module 6 comprises a tensioning cage 62 in a sleeve 60. Slots 63 are provided at a forward facing portion of the tension cage 62 being configured to engage the ball shaped head 32 in a pivotally manner. At its front end, the sleeve 60 comprises a skirt 67 having a rim 66 at its free end. The tension cage 62 is at a forward position in the interior of the skirt 67, and the ball shaped head 32 is moved into engagement by the tension cage 62 through an opening defined by the rim 66. Further, a saddle like cutout 68 is formed at a rear end of the tension cage 62, that saddle like cutout being configured as a receptable for the elongated carrier element 5. From the back end of the sleeve 60, a pressing element 65 is to be mounted. To this end, the sleeve 60 is provided with an inner thread lining the nearly cylindrical wall of the interior of the sleeve 60. The pressing element 65 features a corresponding outer thread on its circumference which engages the inner thread. Thereby, the pressing element 65 moves forward by screwing it in and bears on the tension cage 62 which exerts a clamping force on the ball shaped head 32 of the transfacetal fastening means 3 and on the elongated carrier element 5. Thereby, the transfacetal fastening means 3 is affixed in its angular position relative to the retainer module 6 and provides additional stability by forming a rigid connection to the second fastening means 4 held in the retainer module 6′ farther upward.

For a proper attachment of the elongated carrier element 5 to the sleeve 60, the latter is provided with two opposing slots 63 in a wall of the sleeve. The slots 63 are of such a width to allow a passage of an end portion of the elongated carrier element 5. Thereby, the jump bar 40 passes transversely through the interior of the sleeve 60, between the pressing element 65 and the tension cage 62. In order to give a more rigid fixation, a rear face of the tension cage 62 features a concave portion forming a saddle 68. It is dimensioned such as to provide a form fit for the elongated carrier element 5. The pressure force exerted by the pressing element 65 is thus transmitted via the elongated carrier element 5 to the tension cage 62. Thereby both, the elongated carrier element 5 as well as the tension cage 62 are receiving said pressure force and are locked in their respective positions.

The second fastening means 4 is affixed similarly, although usually to a pars section of the upper vertebra 93. The retainer module 6′ for the second fastening means 4 is configured and employed similar to the retainer module 6.

The length of the elongated carrier element 5 is equivalent to the height of a vertebra 92. As a result, an effect of inserting the elongated carrier element 5 is that the retainer module 6′ is positioned so far upwards that it engages the over next vertebra 93, leaping one intermediate vertebra 92. Thereby, a truly multi-level stabilization is achieved, wherein the stabilizing upward extension attaches to a vertebra 93 which is two levels above that vertebra 91 to which the retainer module 6 is attached.

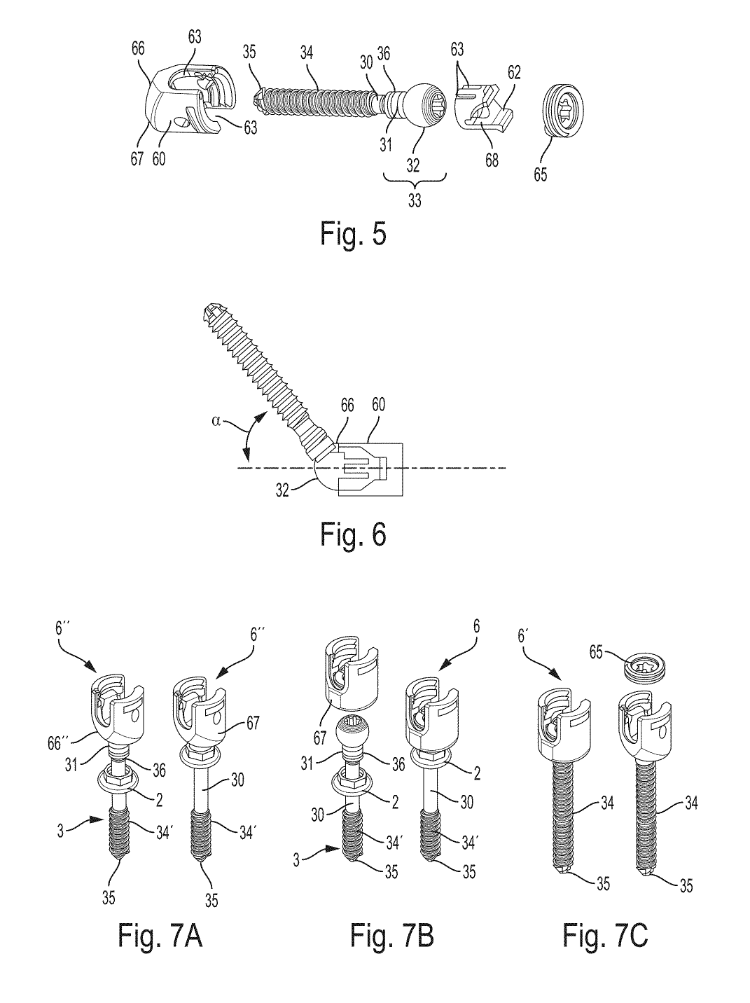

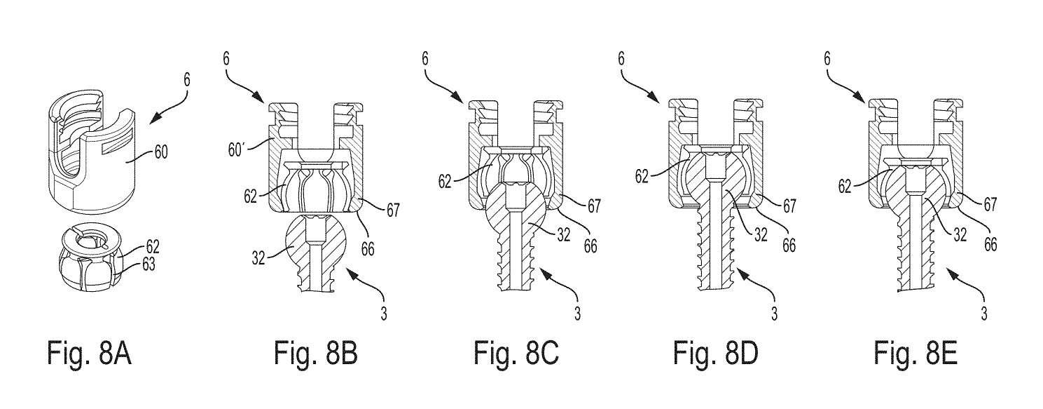

On a front end of the sleeve 60, the circumferential skirt 67 is provided. It delimits with its rim 66 angular movement of the cortical screw of elongated carrier element 5. A variant 6″ as exemplary shown in FIG. 5a features a slanted rim 66′. The length of the skirt 67 is not uniform, rather it is shortest or zero toward a top position and longest toward a bottom position. As a result, the rim 66′ is slanted against a center axis 69 of the sleeve 60. By virtue of this, the upward fixation element can reach a rather steep upward pointing position, up to an angle γ of 50 degrees or even more, as opposed to a much limited movement in a downward direction.

For improved stabilization of the spine, a mirror-shaped implant 1′ is provided. It is configured to be mounted on a side contralateral to the implant 1, as best seen in FIGS. 2b) and c). As it can be readily appreciated from these drawings, the implant 1′ is arranged slanted in relation to implant 1 such that the transfacetal fastening means 3 of both implants 1, 1′ converge at an angle β.

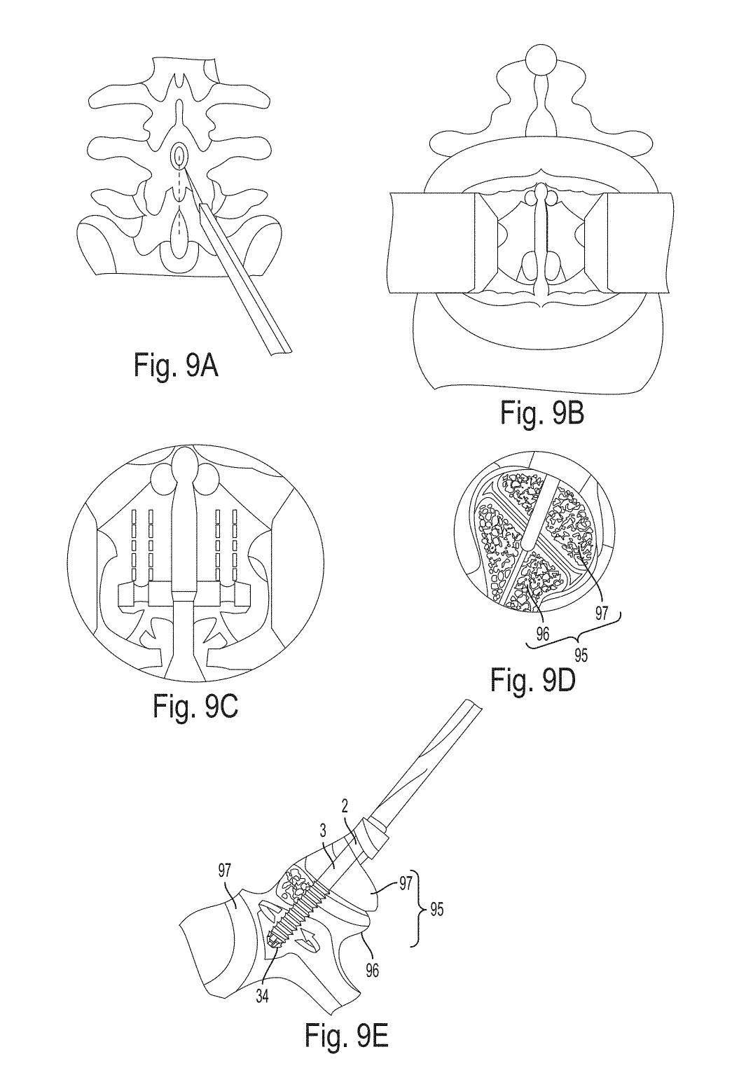

Steps for implanting the implant according to the invention are (i) opening an access hole by cutting and spreading (FIG. 9a, b), then using a template to determine the position (as indicated by the dashed lines in FIG. 9c). A hole is formed through the facet joint 95, the hole passing through the upper section 97 and ending in the lower section 96. The portion of the hole in the upper section may be formed with a bigger drill, thereby providing a width of the hole bigger than an outer diameter of the full thread 34 in the upper section 96 (FIG. 9d, part view on the right hand side shown only). The transfacetal fastening means 3 will be tightened by using a screwdriver of another appropriate tool, thereby pressing the washer 2 down, leading to an immobilization of the facet joint and to a fixation of the polyaxial position of the transfacetal fastening means 3. Similar steps of drilling a hole in a pars section of an upper vertebra and placing the second fastening means 4 therein are performed, using the same compact access hole (see FIG. 1). After completing the implant 1 (see FIG. 2), the access hole is finally closed.

1. A multi-level intersegmental stabilization implant for a facet joint of a first vertebra, the implant comprising a lower retainer module having a transfacetal fastener, the transfacetal fastener having a head and a shaft configured for traversing upper and lower sections of the facet joint, and an upper retainer module comprising a second fastener configured for fastening through a pedicle of a second vertebra, the lower and upper retainer modules being connected by an elongated carrier element dimensioned such as to span across at least two vertebrae,
wherein the transfacetal fastener is configured as a screw having a head and a shaft ending with a tip, the shaft having a thread at least in a region near the tip such that the thread engages exclusively the lower section of the facet joint, and
wherein a washer is connected with the transfacetal fastener, the washer having a hole for the transfacetal fastener, a pressure side, and an opposing seating side, the hole being configured for receiving the shaft of the screw, the pressure side being configured for bearing on an outer surface of the upper section of the facet joint, and the seating side being configured to abut the screw such that a compression force exerted by the screw is transferred through the washer onto the facet joint, wherein the washer provides a polyaxial seat for the screw.

2. The implant of claim 1, wherein at least one of the upper and lower retainer modules is configured as a polyaxial joint configured to hold at an adjustable angle.

3. The implant of claim 1, wherein the transfacetal fastener and the second fastener are angled such that trajectories of the fasteners are converging in a direction away from the vertebrae when the implant is implanted.

4. The implant of claim 1, wherein the shaft of the transfacetal fastener is threaded in a tipward portion only.

5. The implant of claim 1, wherein the shaft of the transfacetal fastener is threaded in both tipward and headward portions.

6. The implant of claim 1, wherein the elongated carrier element is dimensioned such as to bridge an intermediate vertebra between the first and second vertebrae preferably being shorter than a distance between the first and second vertebrae.

7. The implant of claim 6, wherein the elongated carrier element is shaped rodlike and a non-circular cross-section.

8. The implant of claim 1, wherein the retainer module comprises a lockable polyaxial joint.

9. The implant of claim 8, wherein the lockable polyaxial joint comprises a sleeve, a tension cage in an interior, and a pressing element, wherein the tension cage is configured to tiltably engage the head and the pressing element is configured to squeeze the tension cage for arresting of the head.

10. The implant of claim 9, wherein the sleeve has a skirt surrounding a head portion, wherein a rim of the skirt (67) limits a tilting angle of the elongated carrier element.

11. The implant of claim 10, wherein the rim is slanted to a center axis of the sleeve thereby allowing a greater tilt angle in a direction facing away from the elongated carrier element.

12. The implant of claim 9, wherein the sleeve comprises at least one slot in a rear portion, the at least one slot being configured for reception of the elongated carrier element.

13. The implant claim 1, wherein the upper and lower retainer modules are identical.

14. The implant of claim 1, further comprising a second washer for the upper retainer module.

15. The implant of claim 1, wherein the second fastener has a threaded portion configured for an oblique fastening to a cortical structure.

16. The implant of claim 1, wherein the transfacetal fastener further comprises a super-head that together with the head forms a stacked double-head arrangement.

17. The implant of claim 16, wherein the super-head is ball shaped and the head is conically shaped.

18. The implant of claim 16, wherein the super-head is polyaxially held in the sleeve.

19. A set of implants comprising the implant of claim 1, and a second implant is configured as a mirror image for bilateral application on either side of a spine.

20. The set of claim 19, wherein the second implant is arranged configured for implanting in a slanted position such that fasteners of the second implant converge with respect to fasteners of an implant implanted on the other side of the spine.

21-24. (canceled)

25. A method for stabilizing a facet joint, the method comprising:
opening an access hole providing access to a vertebra having a facet joint to be stabilized,
forming a hole through the facet joint, the hole crossing an upper section of the facet joint and reaching into a lower section of the facet joint,
positioning a washer on top of the hole,
inserting a transfacetal fastener through the washer into the hole, tightening the transfacetal fastener such that the facet joint is immobilized, wherein a shaft of the transfacetal fastener is polyaxially orientable with respect to the washer,
forming a second hole in a pars portion of a superior vertebra,
inserting a second fastener, and
closing the access hole,
wherein the transfacetal fastener and the second fasteners are each held in a retainer module, and the retainer modules are connected by a rigid, axially adjustable connection member, the transfacetal fastener being polyaxially adjustable in the retainer module for the transfacetal fastener.

26. The method of claim 25, wherein the connection member spans over at least one intermediate vertebra such that the intermediate vertebra is untouched apart from having an inferior facet fastened.

27. The method of claim 25, wherein the transfacetal fastener and the second fastener are angled such that respective tips of the fasteners diverge by an angle of at least 30°.

28. The method of claim 27, wherein a spacing between the retainer modules of the transfacetal fastener and the second fastener is a distance of between 0.85 and 1.25 of a body height of one vertebra.

29. The method of claim 25, wherein the access hole is at most two times a vertebrae height for a multi-stage implant involving three vertebrae.

30. The method of claim 25, wherein the transfacetal fastener is a screw having a lag thread.

31. The method of claim 25, wherein the transfacetal fastener is a screw having a full thread.

32. The method of claim 31, wherein a bore in the upper section is made wider than a diameter of the full thread.

33. The implant of claim 3, wherein the trajectories converge at an angle of at least 60°.

34. The implant of claim 6, wherein the elongated carrier element is shorter than a distance between the first and second vertebrae.

35. The implant of claim 34, wherein the elongated carrier element is shorter by at least half of a height of a vertebra.

36. The implant of claim 2, wherein the adjustable angle is oblique with respect to the elongated carrier element.

37. The implant of claim 12, wherein the pressing element is configured for arresting the elongated carrier element.
